(19) United States
(12) Patent Application Publication
(10) Pub. No.: US 2019/0021627 A1
(43) Pub. Date: Jan. 24, 2019

(54) INTRACRANIAL BIOIMPEDANCE MEASUREMENT

(71) Applicant: CEREBROTECH MEDICAL SYSTEMS, INC., Pleasanton, CA (US)

(72) Inventors: Mitchell Elliott LEVINSON, Pleasanton, CA (US); Eugene Mark SHUSTERMAN, Pleasanton, CA (US); William Leslie SHEA, Martinez, CA (US)

(21) Appl. No.: 16/043,666

(22) Filed: Jul. 24, 2018

Related U.S. Application Data
(60) Provisional application No. 62/536,155, filed on Jul. 24, 2017.

(51) Int. Cl. A61B 5/053; A61B 5/00; A61B 5/0205

(52) U.S. Cl. CPC A61B 5/0537; A61B 5/6803; A61B 3/16; A61B 5/4064; A61B 5/0205; A61B 5/7246

(57) ABSTRACT

A method for assessing a patient may involve performing a first diagnostic procedure on the patient. The first diagnostic procedure may involve securing a volumetric integral phase-shift spectroscopy (VIPS) device to the patient's head, measuring an intracranial bioimpedance with the VIPS device, and detecting an asymmetry based on the measured intracranial bioimpedance. The method may further involve performing a second diagnostic procedure on the patient using an additional diagnostic device, receiving first patient data from the first diagnostic procedure and second patient data from the second diagnostic procedure in a computer processor, processing the first patient data and the second patient data with the computer processor, and generating an assessment of the patient with the computer processor, based at least in part on the processed first patient data and second patient data.

### CROSS-REFERENCE TO RELATED APPLICATIONS

This application claims priority to U.S. Provisional Patent Application Ser. No. 62/536,155, titled “Intracranial Bioimpedance Measurement, and filed Jul. 24, 2017, the full disclosure of which is hereby incorporated by reference.

This application also incorporates the following U.S. Patents and Applications by reference: U.S. Pat. No. 8,731,636; U.S. Patent Application Pub Nos.: 2013/0190599; 2014/0249400; 2015/0223722; 2015/0374292; 2017/0055839; and 2017/0127946; and U.S. patent application Ser. No. 15/410,838, filed Jan. 20, 2017, titled “Differentiation of Fluid Volume Change,” and Ser. No. 15/635,986, filed Jun. 28, 2017, titled “Continuous Fluid Monitoring System.” All of the above-referenced patents and applications are incorporated by reference in their entireties and are referred to collectively in this application as the “Incorporated References.”

### TECHNICAL FIELD

This application is related to noninvasive, diagnostic, medical devices, systems and methods that use volumetric integral phase-shift spectroscopy (VIPS) to detect or measure intracranial bioimpedance, changes in bioimpedance and/or bioimpedance asymmetry.

### BACKGROUND

In many different medical settings, it would be advantageous to be able to detect, monitor and/or measure intracranial bioimpedance, changes in bioimpedance and/or bioimpedance asymmetry, in a noninvasive manner. For example, it is often critical to detect, measure and/or monitor changes in intracranial fluid content or distribution in an intensive care unit (ICU) patient. Unfortunately, no continuous, noninvasive measurement techniques are currently commercially available for measuring intracranial bioimpedance. Furthermore, many brain injuries are not severe enough to warrant drilling a hole in the cranium for continuous invasive monitoring. Thus, for many patients with brain injury, there is no continuous monitoring technology available to alert clinical staff when there is a potentially harmful increase in edema or bleeding or that a secondary brain injury has occurred. Instead, these patients are typically observed over time by nursing staff. It is not until changes in the fluid composition, volume or distribution in the brain result in observable brain function impairment that the physicians or nurses can react. In other words, there is no way currently available for detecting, monitoring and/or measuring intracranial bioimpedance, changes in bioimpedance and/or bioimpedance asymmetry.

Volumetric integral phase-shift spectroscopy (VIPS) has been previously proposed for diagnosis of brain fluid abnormalities. (VIPS may alternatively be referred to by other acronyms, such as magnetic induction phase-shift spectroscopy (MIPS).) Patents have been awarded for proposed devices, and promising scientific studies of prototype devices are described in the literature. For example, Rubinsky et al. described the use of VIPS for this purpose, in U.S. Pat. Nos. 7,638,341, 7,910,374 and 8,101,421, the disclosures of which are hereby incorporated in their entirety herein. Wyeth et al. described additional details of the use and design of VIPS devices in U.S. Pat. No. 8,731,636, which is hereby incorporated in its entirety herein.

Despite advances made thus far, it would still be advantageous to have improved systems and methods for detecting, monitoring and/or measuring intracranial bioimpedance, changes in bioimpedance and/or bioimpedance asymmetry.

### BRIEF SUMMARY

The present application describes a number of different embodiments for using VIPS technology to detect, monitor and/or measure intracranial bioimpedance, changes in bioimpedance and/or bioimpedance asymmetry, in conjunction with one or more other technologies and/or medical or surgical methods. The ability to quickly ascertain the health of the brain with a non-invasive technology that measures intracranial bioimpedance and/or bioimpedance asymmetry can be used to efficiently and effectively triage a patient to the appropriate level of care. This is important, because any delay in getting correct treatment comes at the cost of the survival of the brain and ultimately the overall outcome of the patient. In some embodiments, for example, the VIPS technology may be used with an additional physiological monitoring technology or technique to enhance the overall patient assessment abilities of the VIPS technology and/or the additional technology, thus providing beneficial information to the physician or other caregiver. In other embodiments, the VIPS technology may be used to enhance the safety of a procedure, such as a surgical or medical procedure. Some embodiments combine the use of VIPS for intracranial bioimpedance measurement with another neurological or intracranial measurement technology, to better assess brain health. In other embodiments, VIPS may be used for intracranial assessment, and the additional technology may assess a different part of the body, such as the heart.

Since the VIPS technologies discussed in this application are fully described in the Incorporated References, they will not be described in this application. Any of the aspects and embodiments described in the Incorporated References may be implemented in the various embodiments described in the present application. Additionally, the terms “VIPS technology” and “VIPS device” are meant to encompass any of the devices and technologies described in the Incorporated References.

In one aspect of the present disclosure, a method for assessing a patient involves performing a first diagnostic procedure on the patient, which includes securing a volumetric integral phase-shift spectroscopy (VIPS) device to the patient's head, measuring an intracranial bioimpedance with the VIPS device, and detecting an asymmetry based on the measured intracranial bioimpedance. The method further involves performing a second diagnostic procedure on the patient using an additional diagnostic device. Next, the method involves receiving first patient data from the first diagnostic procedure and second patient data from the second diagnostic procedure in a computer processor, processing the first patient data and the second patient data with the computer processor, and generating an assessment of the patient with the computer processor. The assessment is based at least in part on the processed first patient data and second patient data.

In various embodiments, any suitable device or combination of devices may be used along with the VIPS device. Examples of the additional diagnostic device include, but are not limited to, an automated external defibrillator, a trans-cranial Doppler device, a diffuse optical tomography device, a heart-rate sensor, an accelerometer, a sound pressure sensor, a brain oxygen sensing device, an intracranial pressure measurement device, an ultrasound device, an intraocular pressure measurement device and a kidney dialysis machine.

In various embodiments, measuring the intracranial bioimpedance may involve measuring an overall intracranial bioimpedance, detecting intracranial bioimpedance asymmetry, and/or measuring a change in intracranial bioimpedance over time. The change in biompedance may be caused by any number of factors, and in some embodiments, the change may be measured after a therapy is provided to the patient, such as the administration of a pharmaceutical agent, a medical procedure, a surgical procedure and/or the like. In some embodiments, the intracranial bioimpedance may be measured during a surgical procedure. In some embodiments, processing the first data and the second data involves combining at least some of the first data with at least some of the second data. For example, the data may be combined via an algorithm embedded in a computer readable medium on the computer processor. In some embodiments, the computer processor is part of the VIPS device. Alternatively, the computer processor may be part of a separate device apart from the VIPS device and the additional diagnostic device.

In another aspect of the present disclosure, a system for assessing a patient may include a first diagnostic device including a volumetric integral phase-shift spectroscopy (VIPS) device, a second diagnostic device, a computer processor linked to the first diagnostic device and the second diagnostic device. The computer processor is configured to receive and process data from the first diagnostic device and the second diagnostic device to provide a patient assessment. According to various embodiments, the second diagnostic device may be any suitable diagnostic device, such as but not limited to those listed above.

In some embodiments, the computer processor is part of the VIPS device. For example, in some embodiments, the VIPS device includes a headset that fits on the patient's head, and the computer processor is located in the headset. In alternative embodiments, the computer processor is part of a separate device apart from the VIPS device and the second diagnostic device. In some embodiments, the system may further include a computer readable medium located on the computer processor and containing instructions for performing a method. The method may involve receiving first patient data from the first diagnostic device, receiving second patient data from the second diagnostic device, combining the first patient data and the second patient data, and processing the combined data to provide the assessment of the patient.

These and other aspects and embodiments will be described in further detail below, in reference to the attached drawing figures.

### BRIEF DESCRIPTION OF THE DRAWINGS

FIG. 1 is a block diagram of a system for assessing a patient, including a VIPS device and an additional device, according to one embodiment; and

FIG. 2 is a flow diagram of a method for assessing a patient, using a VIPS device and an additional device, according to one embodiment.

### DETAILED DESCRIPTION

The VIPS technologies described in the Incorporated References provide for the detection of bioimpedance, bioimpedance changes, and/or bioimpedance asymmetry within the intracranial space of a human or animal subject. The measured bioimpedance is caused by intracranial fluids and tissue. Bioimpedance changes and/or asymmetry may be caused, for example, by a reduction in blood flow (e.g., stenosis, stroke, large vessel occlusion) or destruction of tissue (e.g., ischemic stroke, necrosis, edema). Although VIPS technologies alone are often highly efficacious diagnostic tools, in some cases it might be advantageous to use a VIPS technology in conjunction with one or more additional diagnostic technologies and/or one or more therapeutic technologies. The combination of two or more diagnostic technologies, for example, may help enhance the diagnostic assessment of the patient and lead to improved clinical evaluation, diagnosis and therapy of the patient.

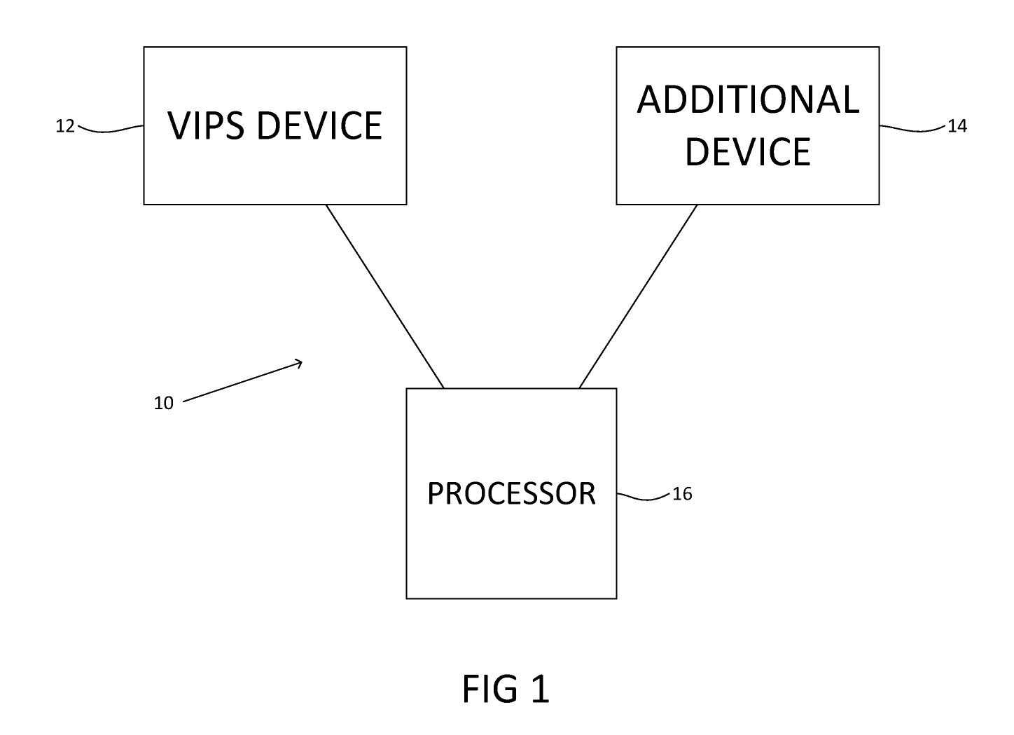

Referring now to FIG. 1, an exemplary diagnostic system 10 may include a VIPS device 12, an additional device 14, and a computer processor 16 coupled to both. In some embodiments, the additional device 14 may be a diagnostic device, while in other embodiments it may be a therapeutic device or a device that performs both diagnostic and therapeutic functions. An automated external defibrillator (AED) is one example of such a device. Although the following description focuses on examples where the additional device 14 is a diagnostic device, this should not be interpreted as limiting the scope of the present application. As shown in FIG. 1, both the VIPS device 12 and also the additional device 14 are connected to the computer processor 16. The computer processor 16 may be part of the VIPS device 12, such as located in a headset that fits on the patient's head or in a console that is part of the VIPS system. Alternatively, the processor 16 may be its own separate device or may be part of the additional device 14. The VIPS device 12 and the additional device 14 may be connected with the processor 16 wirelessly or via wired connections, in various embodiments. In some embodiments, the VIPS device 12 and the additional device 14 may also be directly connected to each other via wired or wireless connection. In other embodiments, the additional device 14 may be incorporated into the VIPS device 12. Some examples of the additional device 14 include, but are not limited to, AEDs, trans-cranial Doppler devices, diffuse optical tomography devices, heart-rate sensors, accelerometers, sound pressure sensors, brain oxygen sensing devices, intracranial pressure measurement devices, ultrasound devices, intraocular pressure measurement devices, and kidney dialysis machines. At least some of these examples are described in further detail below.

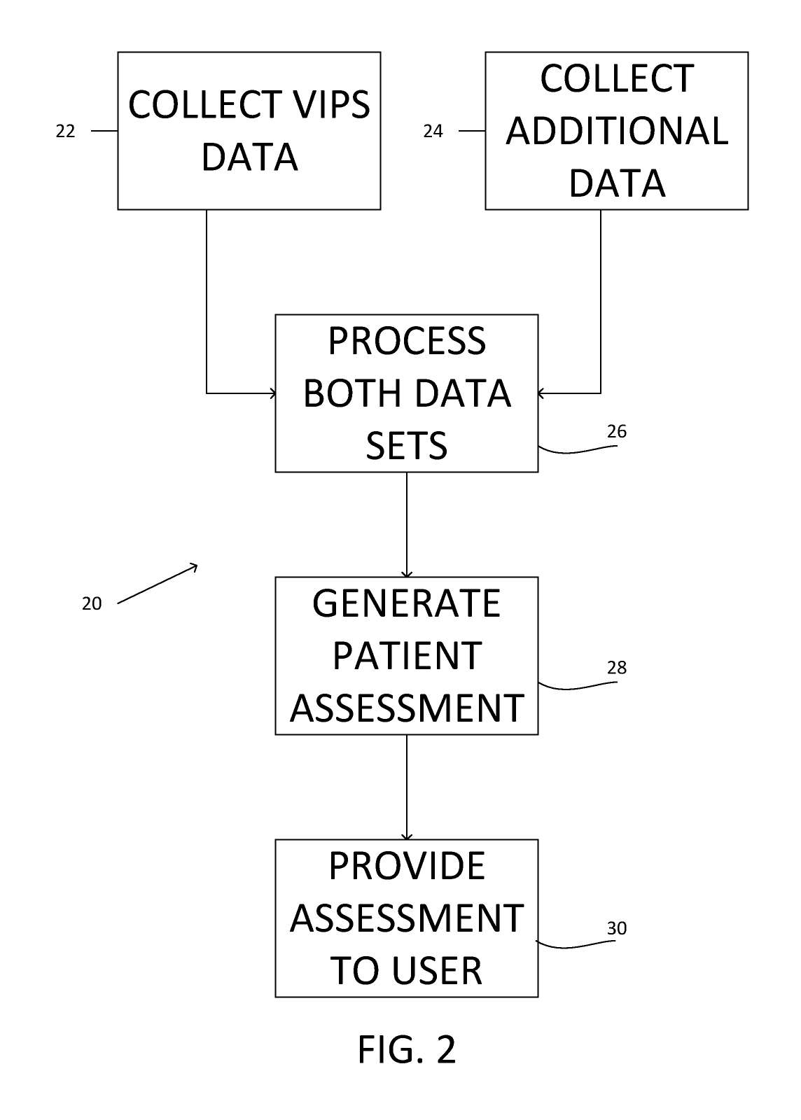

Referring now to FIG. 2, in one embodiment, a method for assessing a patient 20 first involves collecting VIPS data 22, using a VIPS device/system as described fully in the Incorporated References, and collecting additional data 24, using at least one additional diagnostic and/or therapeutic device/system. These two (or more) sets of data may be collected in any order or time sequence. Next, the data sets are received and processed 26 in the computer processor 16. In some embodiments, this step may involve receiving and processing more than two data sets from more than two devices. For example, in some embodiments two additional diagnostic devices may be used, in addition to the VIPS device. The processing step 26 may include combining data from the two or more data sets and/or any other suitable processing, for example using an algorithm embodied in a computer readable medium in the computer processor 16.

Next, the computer processor 16 may generate a patient assessment 28. This assessment may be in any suitable format, such as words, numbers, graphs, charts, alerts, sounds or the like. In general, the assessment describes a patient condition, diagnosis, physiological measurement or similar information, which will allow a physician, nurse or other user to determine the health or a specific condition of the patient. Finally, the method 20 may involve providing the assessment to a user 30. Again, the assessment may be in any form, such as a readout on a screen with letters, words, numbers, graphs, charts, etc. In some embodiments, for example, a console housing the computer processor 16 and having a screen may provide information regarding the patient assessment via a readout on the screen. In another embodiment, for example where the computer processor 16 is located in a VIPS headset that fits on the patient's head, providing the assessment 30 may involve sending data to a separate console with a viewing screen. The method 20 may be repeated as many times and as often as desired for any given patient.

As mentioned above, the additional device 14 (or additional devices) may include any of a number of diagnostic and/or therapeutic devices. Some examples of how such additional devices 14 may be used with a VIPS device/system are described below. These examples should not be interpreted as being the only possible additional devices 14, however, since in alternative embodiments any other suitable device(s) may be used.

In one embodiment, for example, VIPS technology may be used in conjunction with an automated external defibrillator (AED). In this context, VIPS may be used to detect a change (or changes) in intracranial fluid caused by chest compressions. For example, data provided by the VIPS device may provide feedback on the effectiveness of chest compressions, such as the “depth” of compressions, which corresponds to a bioimpedance change measured on the VIPS device. In another embodiment, the VIPS device may detect changes in bioimpedance caused by a transition from hypoxia to normal oxygenation and blood flow, after successful resuscitation of the patient. The VIPS device may also be used to monitor the progression of bioimpedance change over time, such as before, during and after resuscitation. Such a VIPS device may include asymmetry/stroke detection as an AED module.

In another embodiment, VIPS technology may be combined with trans-cranial Doppler (TCD) to better assess a patient's intracranial status. TCD measures cerebral blood flow velocity in the major intracranial arteries by detecting the sound wave change caused by the speed of the red blood cells. TCD may be used to detect emboli, stenosis, vasospasm from subarachnoid hemorrhage, and other abnormalities. In some embodiments, TCD may be performed on each side of the head to measure asymmetry of blood flow in the arteries on each side. A VIPS device can also detect changes in overall bioimpedance caused by any of a number of intracranial irregularities. Combining a VIPS device with a TCD device may improve the accuracy of the information derived from the patient and used by doctor in management of the patient. In addition, the VIPS device may be used to detect bioimpedance asymmetry of the hemispheres of the brain (left vs. right). The VIPS device may be used to detect not only bioimpedance changes caused by a reduction or increase of blood flow (e.g., occlusion, ischemia, hyperemia), but also the effect on an area of the brain. For example, edema or cerebral infarction may be detected.

In yet another embodiment, one or more of the VIPS technologies described in the Incorporated References may be combined or used in conjunction with the Cephalogics diffuse optical tomography (DOT) system for imaging the brain (www.cephalogics.com), or a similar device. The DOT system uses high-density arrangement of near-infrared sources and detectors for spatial measurements to map oxygen saturation and perfusion in the brain, providing imaging of multiple cerebral vascular regions. The VIPS device may be used to detect changes in bioimpedance throughout the brain and the entire intracranial space, which may provide more comprehensive information about blood perfusion deficits in the brain—i.e., not simply the outer surface of the brain. As mentioned, VIPS may also be used to detect spatial (left vs. right hemisphere) bioimpedance differences. The VIPS device may detect bioimpedance changes caused by oxygen saturation/perfusion. Combining and correlating information from a VIPS device and a Cephalogics/DOT device may improve the overall confidence in the diagnosis and insight into the particular pathology at work, and the combination may also provide improved spatial sensitivity.

In another embodiment, the VIPS technology may be used in combination with Jan Medical's BrainPulse™ system (www.janmedical.com), which uses a heart-rate sensor, a sound pressure level sensor (ambient noise), and 6 accelerometers to detect the acceleration of the skull in response to brain oscillation, or a similar device. The BrainPulse™ system is typically used to detect concussion and vasospasm. The VIPS device may provide a complimentary spectrum of data, such as bioimpedance changes correlated to acceleration (whole brain and left vs. right hemisphere) or vasospasm and monitoring bioimpedance changes over time for assessing impacts caused by progression of concussion degradation or vasospasm. VIPS technology may also include integrated heart rate detection, as described in the Incorporated References, so in some cases both the VIPS device and the BrainPulse™ system could be used to monitor heart rate, or the detected heart-rate “pulse” may be used to synchronize the devices' data collection/sampling.

In another embodiment, the VIPS device may be used in combination with Forest Devices' AlphaStroke device (www.forestdevices.com), or a similar device. The AlphaStroke device uses electrodes placed on the head and neck to measure asymmetry in brain oxygen within a minute. The VIPS device may be combined with this technology to enhance the detection of intracranial or brain asymmetry, as discussed above.

In another embodiment, the VIPS technology may be combined with technology developed by HeadSense, Inc. (for example, the HeadSense HS-1000F noninvasive brain assessment modality, www.head-sense.med.com), or similar technology. HeadSense uses ultrasound delivered from an earbud worn by the patient to measure ICP, detect hemorrhagic stroke and the like. This technology may be used with the VIPS technology, which integrates bioimpedance throughout the entire intracranial space. Combining the two technologies should provide better diagnostic capabilities than are possible with the HeadSense device alone.

The VIPS device may also be used in conjunction with the VittaMed non-invasive ICP measurement device. This device uses ultrasound Doppler to scan blood flow parameters in the ophthalmic artery by applying slight pressure to the eye orbit and measuring two segments of the ophthalmic artery (intra-cranial and extra-cranial). It is a non-invasive cerebral autoregulation device that measures ultrasound acoustics from one side of head to other—i.e., “time of flight.” Similar to the other embodiments described above, the VIPS device may provide additional precision, accuracy and detail to the information provided by such a device.

In another embodiment, the VIPS technology may be used in conjunction with any intraocular pressure (TOP) measurement device or technique. For example, if an TOP device is used on a patient and the measured TOP is high, then the VIPS device may be used to measure bioimpedance. If TOP measurement shows an imbalance, then the VIPS device may be used to test for bioimpedance asymmetry. The VIPS device may also be used to assess status of the brain parenchyma and/or cerebrospinal fluid (CSF), test for glaucoma, etc.

In another embodiment, data from the VIPS device may be combined with data from one of the stroke scales to enhance assessment of a patient. Stroke scales include the NIH Stroke Scale, the Cincinnati Stroke Scale, the Los Angeles Prehospital Stroke Scale (LPASS), the ABCD Score, FAST, and others. In some embodiments, the data may be used side-by-side, while in other embodiments the data may be combined, for example by a computer processor using an algorithm, to provide additional information, an enhanced score, or data in any other useful form for a physician, nurse, patient or other user. In one embodiment, for example, the user may enter into the VIPS device, the stroke scale used and the resultant value. The VIPS device may usethis data as part of the algorithm. The combination may improve the sensitivity and specificity for determining the presence of a stroke, such as an LVO.

For any of the embodiments described above, data from the VIPS technology may be combined with data from another diagnostic technology, to provide enhanced data. In some embodiments, this combination may be accomplished via a processor in the VIPS device itself, for example with the processor running an algorithm to combine the data. In alternative embodiments, a separate computing device with a separate processor may receive the data from the VIPS device and the other technology (or multiple technologies) and combine the data to provide the enhanced data. This separate device may be a mobile computing device (smart phone, tablet, etc.), an application on a mobile device, any other suitable computing device, or a custom device made exclusively for the purpose of analyzing data from the VIPS device and one or more other diagnostic devices. The other diagnostic devices may be any suitable devices, such as any of those listed above or devices such as electrocardiogram machines, pulse oxygenation measuring devices, blood pressure cuffs and/or the like.

In yet another embodiment, the VIPS device may be used to provide feedback on the effectiveness of kidney dialysis, by measuring/monitoring bioimpedance changes caused by changes in cerebral edema or other metabolic changes caused by dialysis that alter bioimpedance. For example, the VIPS device may be used to provide feedback on the percent change in bioimpedance over the course of dialysis. In one embodiment, prior to starting dialysis, a VIPS baseline measurement of intracranial bioimpedance is acquired. As dialysis progresses, the VIPS device is used to take additional measurements of intracranial bioimpedance, to detect changes in one or more parameters, such as edema, during the treatment. At the end of treatment, a final VIPS intracranial bioimpedance measurement may be used as a historical reference for subsequent treatments.

In some embodiments, the VIPS technology may be used to monitor the intracranial bioimpedance in patients during surgeries that have a considerable risk of causing cerebral edema, strokes or other brain or intracranial problems. Changes in intracranial bioimpedance may alert the surgeon of underlying complications, such as but not limited to hypoxia, cerebral edema and stroke.

Yet another way in which the VIPS technology may be used is to track brain shrinkage in a patient over time, for example as part of normal health screening or as part of tracking a patient's disease progression. In general, the human brain shrinks with age. For some people, or perhaps for research purposes or as part of regular yearly checkups, it may be advantageous to use a VIPS device to assess overall intracranial bioimpedance and/or intracranial bioimpedance asymmetry. In addition to tracking normal brain shrinkage, this may also act as a screening technique for more frequently detecting abnormalities at an early stage.

Similarly, VIPS devices may be used to detect any of a huge number of brain abnormalities, in patients suffering from current symptoms or again as a screening tool. For example, VIPS devices could be used to detect brain tumors, hydrocephalus, aneurysms, encephalomalacia, sub-clinical seizures, vasospasms, hyperperfusion and the like. In one embodiment, VIPS technology may be used to diagnose migraine headaches. Migraines are known to cause vasodilation, and thus intracranial bioimpedance may change during and/or before migraine onset. VIPS technology might be used for diagnosis as well as for biofeedback for therapy for migraines or other conditions.

We claim:

1. A method for assessing a patient, the method comprising:
performing a first diagnostic procedure on the patient, the first diagnostic procedure comprising:
securing a volumetric integral phase-shift spectroscopy (VIPS) device to the patient's head;
measuring an intracranial bioimpedance with the VIPS device; and
detecting an asymmetry based on the measured intracranial bioimpedance;
performing a second diagnostic procedure on the patient using a first additional diagnostic device;
receiving first patient data from the first diagnostic procedure and second patient data from the second diagnostic procedure in a computer processor;
processing the first patient data and the second patient data with the computer processor; and
generating an assessment of the patient with the computer processor, wherein the assessment is based at least in part on the processed first patient data and second patient data.

2. The method of claim 1, wherein the first additional diagnostic device is selected from the group consisting of an automated external defibrillator, a trans-cranial Doppler device, a diffuse optical tomography device, a heart-rate sensor, an accelerometer, a sound pressure sensor, a brain oxygen sensing device, an intracranial pressure measurement device, an ultrasound device, an intraocular pressure measurement device and a kidney dialysis machine.

3. The method of claim 1, wherein measuring the intracranial bioimpedance comprises at least one of measuring an overall intracranial bioimpedance, detecting intracranial bioimpedance asymmetry, and measuring a change in intracranial bioimpedance over time.

4. The method of claim 1, wherein the intracranial bioimpedance is measured at least one of before, during or after delivering a therapy to the patient, and wherein the therapy is selected from the group consisting of a surgical procedure, a medical procedure, and administration of a pharmaceutical agent.

5. The method of claim 1, wherein processing the first data and the second data comprises combining at least some of the first data with at least some of the second data.

6. The method of claim 5, wherein the data is combined via an algorithm embedded in a computer readable medium on the computer processor.

7. The method of claim 1, wherein the computer processor is part of the VIPS device.

8. The method of claim 1, wherein the computer processor is part of a separate device, apart from the VIPS device and the first additional diagnostic device.

9. The method of claim 1, further comprising:
performing a third diagnostic procedure on the patient using a second additional diagnostic device;
receiving third patient data from the third diagnostic procedure in the computer processor;
processing the third patient data with the computer processor; and
generating the assessment of the patient with the computer processor, wherein the assessment is based at least in part on the processed first patient data, the processed second patient data, and the processed third patient data.

10. A system for assessing a patient, the system comprising:
a first diagnostic device, comprising a volumetric integral phase-shift spectroscopy (VIPS) device;
a second diagnostic device; and
a computer processor linked to the first diagnostic device and the second diagnostic device, configured to receive and process data from the first diagnostic device and the second diagnostic device to provide a patient assessment.

11. The system of claim 10, wherein the second diagnostic device is selected from the group consisting of a trans-cranial Doppler device, a diffuse optical tomography device, a heart-rate sensor, an accelerometer, a sound pressure sensor, a brain oxygen sensing device, an intracranial pressure measurement device, an ultrasound device, and an intraocular pressure measurement device.

12. The system of claim 10, wherein the computer processor is part of the VIPS device.

13. The system of claim 12, wherein the VIPS device includes a headset that fits on the patient's head, and wherein the computer processor is located in the headset.

14. The system of claim 10, wherein the computer processor is part of a separate device apart from the VIPS device and the second diagnostic device.

15. The system of claim 10, wherein the computer processor is part of the second diagnostic device.

16. The system of claim 10, further comprising a third diagnostic device, wherein the computer processor is linked to the first diagnostic device, the second diagnostic device, and the third diagnostic device and is configured to receive and process data from the first diagnostic device, the second diagnostic device, and the third diagnostic device, to provide a patient assessment

17. The system of claim 10, further comprising a computer readable medium located on the computer processor and containing instructions for performing a method, the method comprising:
receiving first patient data from the first diagnostic device;
receiving second patient data from the second diagnostic device;
combining the first patient data and the second patient data; and
processing the combined data to provide the assessment of the patient.
